(19) United States
(12) Patent Application Publication
(10) Pub. No.: US 2025/0052722 A1
(43) Pub. Date: Feb. 13, 2025

(54) PHOTOACOUSTIC WAVE MEASUREMENT DEVICE AND PHOTOACOUSTIC WAVE MEASUREMENT SYSTEM

(71) Applicant: TOYAMA PREFECTURAL UNIVERSITY, Imizu-shi, Toyama (JP)

(72) Inventors: Isao SHIMOYAMA, Imizu-shi, Toyama (JP); Kentaro NODA, Imizu-shi, Toyama (JP); Takuya TSUKAGOSHI, Imizu-shi, Toyama (JP)

(21) Appl. No.: 18/721,063

(22) PCT Filed: Dec. 23, 2022
(86) PCT No.: PCT/JP2022/047730

§ 371 Date: Sep. 19, 2024

(30) Foreign Application Priority Data
Dec. 24, 2021 (JP) 2021-211732

(51) Int. Cl. G01N 29/24; G01N 29/14; G01N 33/49

(52) U.S. Cl. CPC G01N 29/2418; G01N 29/14; G01N 33/49; G01N 2291/022

(57) ABSTRACT

A photoacoustic wave measurement device includes a modulated light source that irradiates a measurement target with far-infrared light, which is subjected to intensity modulation; and a measurement unit that measures a photoacoustic wave emitted from the measurement target irradiated with the far-infrared light. The measurement unit includes an airtight chamber that is filled with a gas therein and receives the photoacoustic wave emitted from the measurement target; and a pressure sensor constituting a part of a wall portion that partitions the airtight chamber from the outside.

### TECHNICAL FIELD

The present invention relates to a photoacoustic wave measurement device and a photoacoustic wave measurement system.

### BACKGROUND ART

A photoacoustic wave measurement device is known as a device for measuring a molecular concentration inside a measurement target. In a case where the measurement target is irradiated with modulated light, a photoacoustic wave (elastic wave) corresponding to a modulation pattern of the modulated light is generated when an internal molecule absorbs light and instantaneously thermally expands. The photoacoustic wave measurement device can detect the photoacoustic wave and determine a concentration of the molecule that absorbs the modulated light, according to the magnitude of the detected photoacoustic wave. As an example of such a photoacoustic wave measurement device, there is known a device that measures a pressure change in an airtight chamber that changes according to a photoacoustic wave using a pressure sensor and measures a molecular concentration of a measurement target (for example, Patent Literature 1).

### CITATION LIST

### Patent Literature

Patent Literature 1: JP2019-7824A

### SUMMARY OF INVENTION

### Technical Problem

In an aspect of applications of a photoacoustic wave measuring method, a blood glucose concentration of blood flowing through a blood vessel inside the skin can be measured. According to the technique disclosed in Patent Literature 1, near-infrared light having a low absorption coefficient of moisture in blood that may cause noise is used to measure the blood glucose concentration. However, since the absorption coefficient of glucose with respect to near-infrared light is also small, it is necessary to detect the photoacoustic wave after amplifying the photoacoustic wave by a resonator or the like, and there is a problem that the photoacoustic wave measurement device is increased in size.

The present invention has been made to solve such a problem, and an object thereof is to miniaturize a photoacoustic wave measurement device.

### Solution to Problem

A photoacoustic wave measurement device according to an aspect of the present invention includes a modulated light source that irradiates a measurement target with far-infrared light, which is subjected to intensity modulation; and a measurement unit that measures a photoacoustic wave emitted from the measurement target irradiated with the far-infrared light. The measurement unit includes an airtight chamber that is filled with a gas therein and receives the photoacoustic wave emitted from the measurement target; and a pressure sensor constituting a part of a wall portion that partitions the airtight chamber from the outside.

A photoacoustic wave measurement system according to an aspect of the present invention includes a modulated light source that irradiates a measurement target with far-infrared light, which is subjected to intensity modulation; a measurement unit that measures a photoacoustic wave emitted from the measurement target irradiated with the far-infrared light; and a controller that controls the modulated light source and the measurement unit. The measurement unit includes an airtight chamber that is filled with a gas therein and receives the photoacoustic wave emitted from the measurement target; and a pressure sensor constituting a part of a wall portion that partitions the airtight chamber from the outside. The controller irradiates the modulated light source with the far-infrared light, which is subjected to the intensity modulation, acquires a pressure change in the airtight chamber by the pressure sensor, and calculates a concentration of a predetermined component in the measurement target according to the pressure change acquired by the pressure sensor.

### Advantageous Effects of Invention

With the photoacoustic wave measurement device according to an aspect of the present invention, in a case where the measurement target is irradiated with far-infrared light, the photoacoustic wave radiated from the predetermined component (for example, glucose) in the measurement target reaches the airtight chamber. Accordingly, an air pressure in the airtight chamber changes, and the concentration of the predetermined component can be calculated by detecting the change in the air pressure by the pressure sensor. In general, since an absorption coefficient of a component (blood glucose) in an aqueous solution with respect to far-infrared light is high, a change in the air pressure of the airtight chamber can be measured without providing a resonator or the like, and thus the photoacoustic wave measurement device can be miniaturized.

### BRIEF DESCRIPTION OF DRAWINGS

FIG. 1 is a schematic configuration diagram of a photoacoustic wave measurement device according to a first embodiment;

FIG. 2 is a sectional view of a sensor unit;

FIG. 3 is an exploded perspective view of the sensor unit;

FIG. 4 is a graph showing measurement results for glucose solutions having various concentrations;

FIG. 5 is a graph showing the measurement results of FIG. 4 in another aspect.

FIG. 6 is a schematic configuration diagram of a photoacoustic wave measurement device according to a second embodiment;

FIG. 7 is a schematic configuration diagram of a photoacoustic wave measurement device according to a third embodiment;

FIG. 8 is a schematic configuration diagram of a photoacoustic wave measurement device according to a fourth embodiment; and

FIG. 9 is a schematic configuration diagram of a photoacoustic wave measurement device according to a fifth embodiment.

### DESCRIPTION OF EMBODIMENTS

Hereinafter, embodiments of the present invention will be described with reference to the drawings.

### First Embodiment

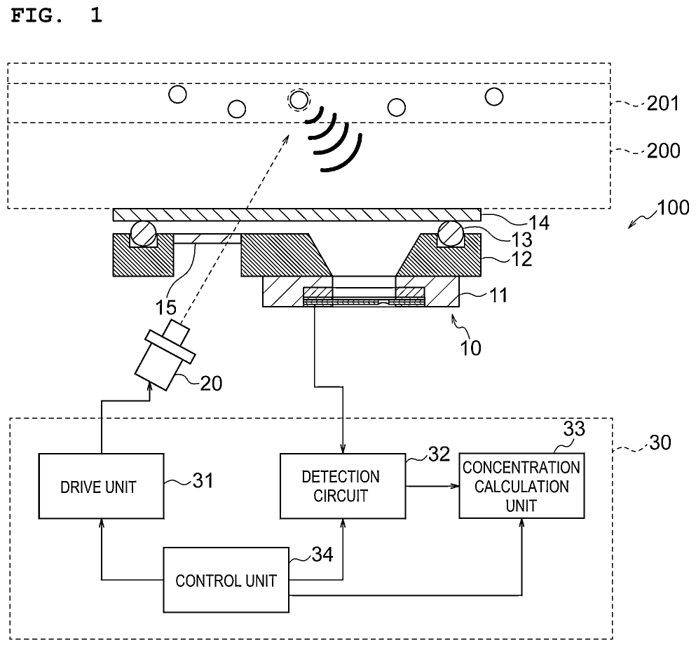

FIG. 1 is a schematic configuration diagram of a photoacoustic wave measurement device according to a first embodiment. In FIG. 1, a photoacoustic wave measurement device (hereinafter, simply referred to as a measurement device) 100 includes a measurement unit 10 that performs photoacoustic wave measurement, a modulated light source 20, and a circuit unit 30. The measurement device 100 can measure a concentration of a predetermined component present in a measurement target 200.

An outline of measurement on the concentration of the predetermined component in the measurement target 200 is as follows. In a case where the measurement target 200 is irradiated with light from the modulated light source 20, a photoacoustic wave (elastic wave) is generated by a photoacoustic effect in molecules in the measurement target 200. The measurement unit 10 detects the photoacoustic wave generated from the measurement target 200. The circuit unit 30 calculates a concentration of a liquid measured in the measurement target 200 according to a change in intensity of the photoacoustic wave measured by the measurement unit 10.

In the example of the present embodiment, the measurement target 200 is a living body, and in a case where the measurement target 200 is irradiated with far-infrared light from the modulated light source 20, a photoacoustic wave is emitted from glucose in blood flowing through a blood vessel 201 in the measurement target 200. The photoacoustic wave is detected by the measurement unit 10, and a blood glucose concentration in the blood vessel 201 is calculated based on a detection result. Hereinafter, each configuration will be described in detail.

The circuit unit 30 includes a drive unit 31, a detection circuit 32, a concentration calculation unit 33, and a control unit 34 that collectively controls these units.

The modulated light source 20 outputs a modulated light of far-infrared light (wavelength of about 10 μm) toward the measurement target 200 under control of the drive unit 31. For example, the modulated light source 20 includes a laser light source for far-infrared light and a chopper part, and the chopper part operates under the control of the drive unit 31, so that the far-infrared light whose intensity is modulated is output as modulated light. In one measurement, a pulsed light is output a plurality of times, for example, about five times. Intensity-modulated light is not limited to a pulsed light in which output of the light is completely blocked, and the light may be modulated to reduce the intensity to a predetermined level (>0), for example. The modulated light source 20 may perform intensity modulation by turning on and off the laser light source at a predetermined cycle without including a chopper part.

In a case where the measurement target 200 is irradiated with the modulated light from the modulated light source 20 and the far-infrared modulated light reaches the blood vessel 201, glucose in the blood absorbs the far-infrared light and instantaneously expands to generate a photoacoustic wave (elastic wave). The photoacoustic wave generates a pressure change in an airtight chamber included in the measurement unit 10, and the pressure change is measured by the sensor unit 11. Here, since a larger photoacoustic wave is generated as the glucose concentration is higher, the concentration calculation unit 33 can calculate the blood glucose concentration according to the intensity of the photoacoustic wave. The concentration calculation unit 33 can remove (reduce) noise and determine the concentration with higher accuracy by synchronizing and associating a modulation pattern of the modulated light source 20 with the pressure change measured by the measurement unit 10.

The measurement unit 10 includes a sensor unit 11, a housing 12, a seal member 13, a contact portion 14, and a window portion 15.

The housing 12 is a bottomed member having a tapered opening whose width decreases toward the outside. The measurement unit 10 is fixed in a state where an end portion on a side surface of the housing 12 which is a bottomed member is pressed against the measurement target 200 via the contact portion 14. Further, the internal space of the housing 12 communicates with an opening provided in the sensor unit 11 and is integrated with a space between the housing 12 and the contact portion 14 to form an airtight chamber. The seal member 13 is provided between the housing 12 and the contact portion 14, so that the degree of adhesion between the housing 12 and the contact portion 14 is increased, and the airtightness of the space (airtight chamber) in the housing 12 is improved. An example of the seal member 13 is an O-ring, and is disposed in a groove portion provided in an end portion on the side surface of the housing 12. The seal member is also referred to as a sealing member.

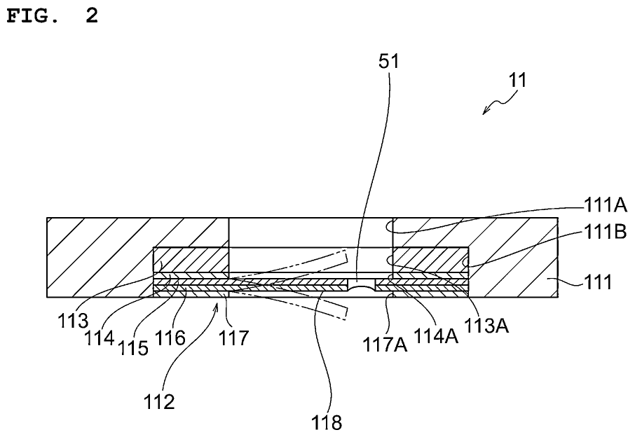

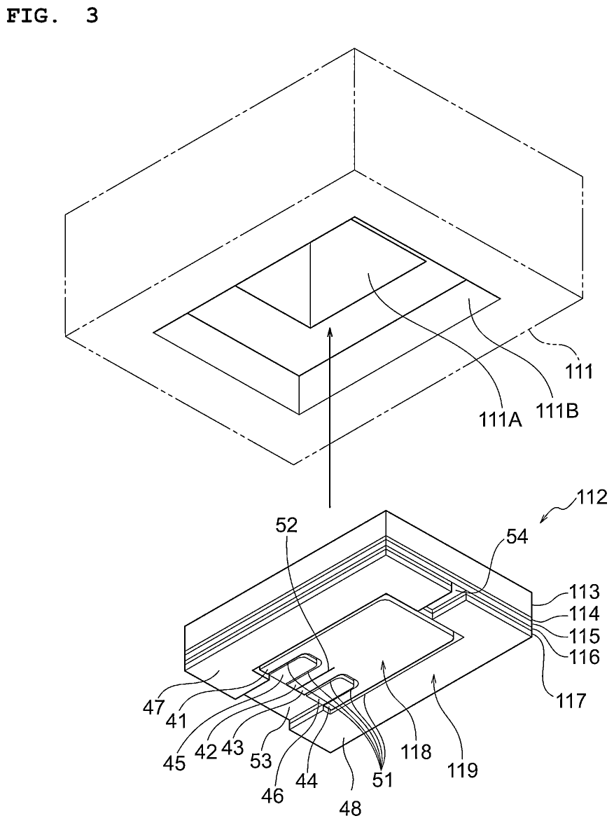

The intensity of the photoacoustic wave can be calculated by measuring, by the sensor unit 11, a change in air pressure in the airtight chamber configured in this manner. A structure of the sensor unit 11 will be described later with reference to FIGS. 2 and 3. In FIG. 1, the housing 12 is a bottomed member having a tapered opening, but is not limited thereto. The housing 12 may have any structure as long as a space that may be an airtight chamber exists therein.

The contact portion 14 is made of an elastic substance that has high transmittance for far-infrared light and is physically easily vibrated. The contact portion 14 is made of, for example, polyethylene having a thickness of 70 μm. The contact portion 14 is preferably made of a material that is displaced following displacement on the surface of the measurement target. Since the displacement due to the influence of the photoacoustic wave may occur even in the measurement target, the influence of the photoacoustic wave in the airtight chamber can be increased by forming the contact portion 14 with an elastic substance, and the measurement accuracy of the predetermined component can be improved.

The window portion 15 is provided in an opening existing in a light guiding path from the modulated light source 20 of the housing 12, and is made of a substance having high transmittance for far-infrared light. The window portion 15 may be made of a substance having high heat resistance so as to withstand a temperature rise caused by direct irradiation with far-infrared light. In another aspect, the window portion 15 may be made of a material that is hardly affected by far-infrared light, for example, without alteration by irradiation with far-infrared light or alteration by absorption of far-infrared light. The window portion 15 may optionally have such a property, and may have at least one of high transmittance, high heat resistance, and high durability of far-infrared light. The window portion 15 is made of, for example, a thin film of silicon or germanium which is resistant to heat. As an example, germanium has a thickness of about 1 mm and is hardly deformed. In general, in a case where the thickness of the material is increased, it is difficult to transmit the far-infrared light, and therefore, the material and the thickness of the window portion 15 are determined by design so that a transmission amount of the far-infrared light is sufficient. When the far-infrared light is sufficiently transmitted, it is preferable that the thickness is large because the influence of the outside air pressure on the airtight chamber can be suppressed. Alternatively, the window portion 15 may be made of polyethylene. Since a thin film of silicon or germanium has a property of being physically easily vibrated, a thin film of silicon or germanium having a thickness of 100 μm or less may be provided in the contact portion 14 as in the window portion 15.

FIG. 2 is an enlarged view of the sensor unit 11. As illustrated in an exploded perspective view of the sensor unit 11 in FIG. 3, in the sensor unit 11, a sensor substrate 112 is fitted and fixed to a fixing member 111 having a stepped opening inside.

Referring again to FIG. 2, the fixing member 111 includes an opening 111A provided on an airtight chamber side and having a relatively small opening area, and an opening 111B provided outside the measurement unit 10 and having a relatively large opening area. The openings 111A and 111B communicate with each other, and a step portion is formed therebetween. The sensor substrate 112 is fitted into the opening 111B and is fixed to the step portion of the fixing member 111 by locking.

The sensor substrate 112 is formed by laminating a silicon substrate 113, an insulating layer 114, a silicon layer 115, a piezoresistive layer 116, and a metal layer 117. The silicon substrate 113, the insulating layer 114, and the metal layer 117 include openings 113A, 114A, and 117A, respectively. The openings 113A, 114A, and 117A have approximately the same opening area as the opening 111A provided on the airtight chamber side of the fixing member 111.

The silicon layer 115 and the piezoresistive layer 116 are integrated, and one end portion (left side in FIG. 2) is sandwiched and fixed between the metal layer 117 and the silicon substrate 113 and the insulating layer 114. A groove portion 51 is provided, so that the other end portion of the silicon layer 115 and the piezoresistive layer 116 (right side in FIG. 2) becomes a free end without being fixed. Furthermore, as illustrated in FIG. 3, the silicon layer 115 and the piezoresistive layer 116 are configured such that a pressure receiving portion 118 at a central portion is rectangular, and are disposed inside a frame 119. The pressure receiving portion 118 is fixed to the frame 119 only at one side (left side in FIG. 2, left lower side in FIG. 3), and the other three sides are free ends that are not fixed to the frame 119. Therefore, the pressure receiving portion 118 is in a cantilever manner. A sensing portion having such a configuration of the pressure receiving portion 118 is generally referred to as a cantilever.

Referring again to FIG. 2, the pressure receiving portion 118 (silicon layer 115 and piezoresistive layer 116) in a cantilever manner is a plate-like member having a thickness of, for example, about 0.3 μm. As a result, the pressure receiving portion 118 is displaced (vibrated) in a vertical direction with respect to the opening according to an air pressure difference between the airtight chamber (openings 111A, 113A, and 114A) and the outside. The piezoresistive layer 116 has a property of increasing or decreasing a resistance value by compression or extension. Accordingly, a change of the airtight chamber caused by the photoacoustic wave emitted from the measurement target 200 can be detected by measuring the resistance value of the piezoresistive layer 116.

FIG. 3 is an exploded perspective view of the sensor unit 11. As illustrated in the drawing, in the sensor unit 11, the sensor substrate 112 is fitted and fixed to the opening 111B of the fixing member 111.

When the sensor substrate 112 is viewed in a lamination direction, the rectangular pressure receiving portion 118 is surrounded by the frame 119 and is in a cantilever manner fixed to the frame 119 only on one side (lower left side). As described above, the pressure receiving portion 118 is fixed to the frame 119 by integrally sandwiching the silicon layer 115 and the piezoresistive layer 116 between the metal layer 117 and the silicon substrate 113 and the insulating layer 114.

Furthermore, when attention is paid to one side (lower left side) where the pressure receiving portion 118 is fixed to the frame 119, the pressure receiving portion 118 is fixed to the frame 119 via four beam portions 41 to 44.

The beam portions 41 and 42 form a pair, and the beam portions 43 and 44 form a pair. The beam portions 41 and 44 are located at two ends, and the beam portions 42 and 43 are adjacent to each other via a slit groove portion 52. Between the paired beam portions 41 and 42 and between the paired beam portions 43 and 44, rectangular protruding portions 45 and 46 protruding from the frame 119 are respectively provided. The protruding portions 45 and 46 include the silicon layer 115 and the piezoresistive layer 116, and are formed integrally with the frame 119. The protruding portions 45 and 46 are separated from the pressure receiving portion 118 via the groove portion 51 on three sides other than a side in contact with the frame 119.

In this manner, the pressure receiving portion 118 is connected to the frame 119 via the beam portions 41 to 44 on one side (lower left side) on which the beam portions 41 to 44 are provided. At the same time, the pressure receiving portion 118 is separated, via the groove portion 51, from the frame 119 at three sides (lower right side, upper right side, and upper left side) where the beam portions 41 to 44 are not provided, and is also separated from the protruding portions 45 and 46. As described above, the slit groove portion 52 is provided between the beam portions 42 and 43. The groove portion 51 and the slit groove portion 52 are formed with a width of, for example, about 0.02 μm to 10 μm.

Furthermore, in the metal layer 117, an insulating groove portion 53 is provided between the beam portions 41 and 44 on a side (lower left side) where the pressure receiving portion 118 is fixed in a cantilever manner, and an insulating groove portion 54 is provided in a center portion on an opposite side (upper right side). The insulating groove portions 53 and 54 are provided to electrically insulate the inside of the metal layer 117, and accordingly, two electrodes 47 and 48 are formed.

The beam portion 41 of the piezoresistive layer 116 is connected to the electrode 47, and the beam portion 44 is connected to the electrode 48. The beam portions 42 and 43 are electrically insulated from the metal layer 117. In the piezoresistive layer 116, a conductive path is formed in a substantially U-shape connected by a path from the beam portion 41 at one end to the beam portion 44 at the other end through the pressure receiving portion 118. Therefore, the resistance value of the pressure receiving portion 118 can be calculated by measuring a current between the electrodes 47 and 48.

In this configuration, the pressure receiving portion 118 is elastically deformable around base ends of the beam portions 41 to 44 in a normal direction (upper-lower direction in drawing) of the surface. Since the beam portions 41 to 44 are curved due to deformation of the pressure receiving portion 118, the resistance value of the piezoresistive layer 116 increases or decreases. As a result, a change in the air pressure of the airtight chamber caused by the received photoacoustic wave can be measured by measuring a resistance between the electrodes 47 and 48.

A method of manufacturing the sensor unit 11 is as follows. First, a silicon on insulator (SOI) substrate in which the silicon substrate 113, the insulating layer 114, and the silicon layer 115 are laminated is prepared. Thicknesses of the silicon substrate 113, the insulating layer 114, and the silicon layer 115 are, for example, 300 μm, 0.4 μm, and 0.08 μm in this order. The silicon layer 115 of the SOI substrate is doped to form the piezoresistive layer 116 on a surface layer of the silicon layer 115. After the metal layer 117 serving as an electrode is formed on the piezoresistive layer 116, the metal layer 117, the piezoresistive layer 116, and the silicon layer 115 are sequentially etched to form the opening 117A, the groove portion 51, and the slit groove portion 52. Furthermore, the insulating groove portions 53 and 54 are provided by etching the metal layer 117, so that the electrodes 47 and 48 are formed. Finally, the silicon substrate 113 and the insulating layer 114 are etched from an opposite surface side to form the openings 113A and 114A. In this manner, the sensor substrate 112 including the pressure receiving portion 118 is formed.

The measurement unit 10 includes, as a reference resistance, a cantilever (not illustrated) having the same configuration as that of the cantilever included in the sensor unit 11. The detection circuit 32 compares a resistance value of the cantilever forming a part of an outer wall of the airtight chamber with a resistance value of the reference resistance to detect a change in the air pressure of the airtight chamber.

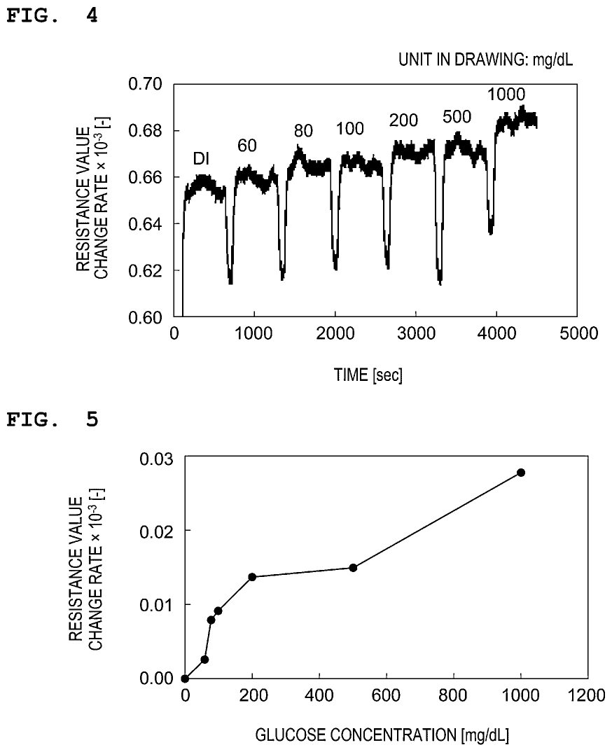

FIG. 4 is a graph showing measurement results by the measurement device 100. In the example of this graph, the modulated light source 20 modulates the far-infrared light emitted from the laser light source by the chopper part driven at 40 Hz, and irradiates the measurement target 200 with the modulated light.

The measurement target 200 is a glucose solution having various concentrations, and seven kinds of solutions in total are measured: pure water (DI: deionized water) and six kinds of glucose solutions having glucose concentrations of 60 mg/dL, 80 mg/dL, 100 mg/dL, 200 mg/dL, 500 mg/dL, and 1,000 mg/dL. These solutions having a plurality of concentrations are measured after being sequentially replaced in time series, and a drop in the resistance value change rate is observed at the timing of replacement.

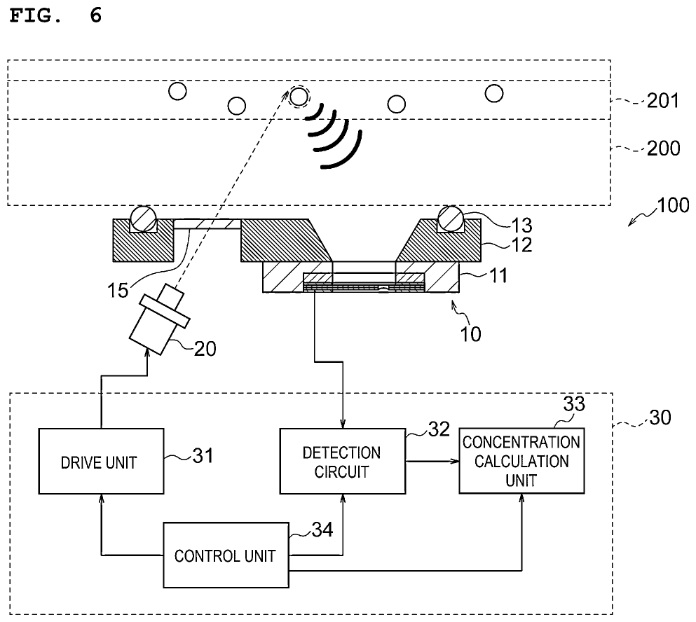

FIG. 5 is a graph showing the measurement results of FIG. 4 in another aspect. In this graph, a horizontal axis indicates the glucose concentration, and a vertical axis indicates the resistance value change rate. The vertical axis is offset from a value when the glucose concentration is zero (DI). As shown in this drawing, the resistance value change rate increases as the glucose concentration increases (0 (DI), 60 mg/dl, 80 mg/dL, 100 mg/dL, 200 mg/dL, 500 mg/dl, and 1,000 mg/dL). In general, when the measurable lower limit is 60 mg/dl and the resolution is 20 mg/dL, it is said to be industrially usable, and the measurement result shown in this graph satisfies the required level.

The far-infrared light used in the present embodiment has a large absorption coefficient with respect to moisture and blood glucose as compared with light in a near-infrared range to mid-infrared range (wavelength of about 0.7 μm to 4 μm to 5 μm). Although the absorption coefficient of blood glucose is large with respect to mid-infrared light and near-infrared light, the absorption coefficient of moisture present in a large amount in the measurement target 200 is also large, and thus a large amount of noise may be measured. However, it is indicated by the present embodiment that the photoacoustic wave from the blood glucose may be measured even when far-infrared light is used.

Further, since the absorption coefficient of glucose for the far-infrared light is large, the intensity of the photoacoustic wave is increased by using the far-infrared light. As a result, it is possible to measure a change in the air pressure of the airtight chamber by the cantilever without providing a resonator or the like, and thus it is possible to miniaturize the measurement device 100. An airtight chamber may be provided, and since the pressure change in the airtight chamber is increased by providing the airtight chamber, the measurement accuracy of the measurement device 100 can be improved. A resonator structure may be provided, and the measurement accuracy of the measurement device 100 can be improved by increasing the pressure change in the airtight chamber by the resonator.

In the present embodiment, an example is described in which one measurement unit 10 is provided, but the present invention is not limited thereto, and a plurality of measurement units may be provided. For example, a generation position of the photoacoustic wave can be specified by overlapping frequency bands of the pressure changes respectively measurable by the plurality of measurement units 10. The frequency bands of the pressure changes respectively measurable by the plurality of measurement units 10 are different from one another, so that the vibration corresponding to the measurement target can be detected, and therefore, the concentrations of the plurality of measurement targets can be simultaneously measured.

### Second Embodiment

In the first embodiment, an example is described in which the measurement device 100 is separated from the measurement target 200 via the contact portion 14, but the present invention is not limited thereto. In the present embodiment, an example in which there is no contact portion 14 will be described.

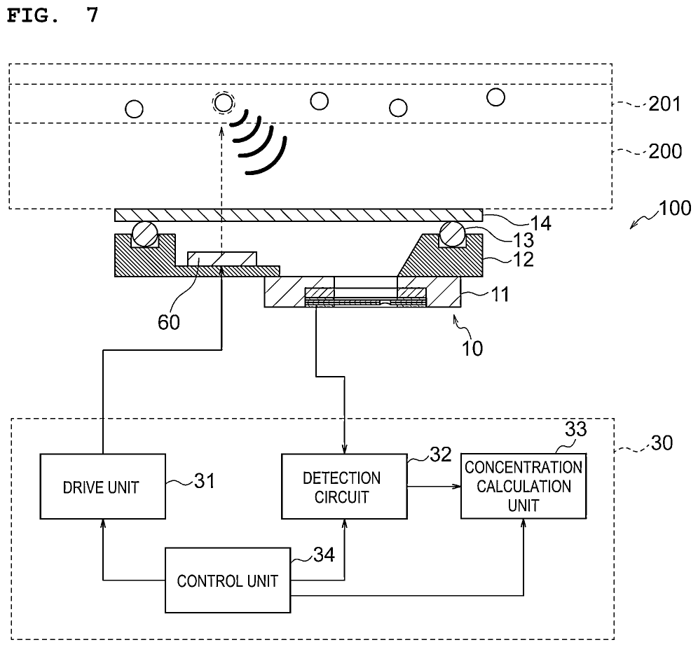

FIG. 6 is a schematic configuration diagram of the measurement device 100 according to a second embodiment, and corresponds to FIG. 1. According to the drawing, the contact portion 14 is eliminated as compared with the measurement device 100 according to the first embodiment.

In such a configuration, in a case where an end portion on a side surface of the housing 12 which is a bottomed member is pressed against the measurement target 200, the housing 12 comes into contact with a surface of the measurement target 200 via the seal member 13. Accordingly, the surface of the measurement target 200 becomes a part of a wall portion of an airtight chamber, and the airtight chamber is formed inside the housing 12.

Since the contact portion 14 is not provided in this manner, there is no intervening substance between a generation source of the photoacoustic wave and the airtight chamber, and the change in the air pressure caused by the photoacoustic wave (elastic wave) in the airtight chamber becomes larger. As a result, the change in the air pressure of the airtight chamber can be easily measured, and therefore, the measurement accuracy of the measurement device 100 can be improved. Furthermore, the structure of the measurement device 100 can be simplified by not providing the contact portion 14.

### Third Embodiment

In the first embodiment, an example is described in which the modulated light source 20 includes a laser light source and a chopper part, but the present invention is not limited thereto. In the present embodiment, an example in which the modulated light source 20 of another aspect is used will be described.

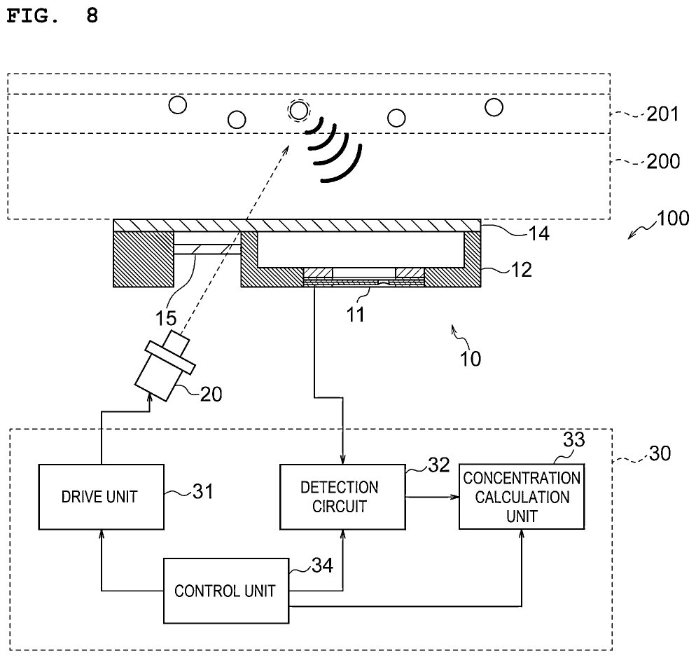

FIG. 7 is a schematic configuration diagram of the measurement device 100 according to a third embodiment, and corresponds to FIG. 1. According to this drawing, a thermal light source 60 is provided as a light source for far-infrared light. In general, it is known that a heat source emits light of a predetermined wavelength according to the temperature.

For example, an electrical resistor is used as the thermal light source 60, and a current flows through the electrical resistor in response to an energization instruction from the drive unit 31 to generate heat, thereby emitting far-infrared light of a predetermined wavelength. The irradiation light is modulated by turning on and off the current. Furthermore, the thermal light source 60 is provided with a temperature sensor, and the temperature of the thermal light source 60 may be notified to the drive unit 31. Feedback control is performed using acquisition by the temperature sensor, so that the drive unit 31 can emit light of a predetermined wavelength by maintaining the thermal light source 60 at a predetermined temperature. With such a configuration, it is possible to emit far-infrared light without using a large device such as a laser light source as in the first embodiment.

### Fourth Embodiment

In the first to third embodiments, examples are described in which in the measurement unit 10, the sensor unit 11 is attached to a wall surface of the housing 12, but the present invention is not limited thereto. In a fourth embodiment, an example in which the sensor unit 11 constitutes a part of the wall surface of the housing 12 will be described.

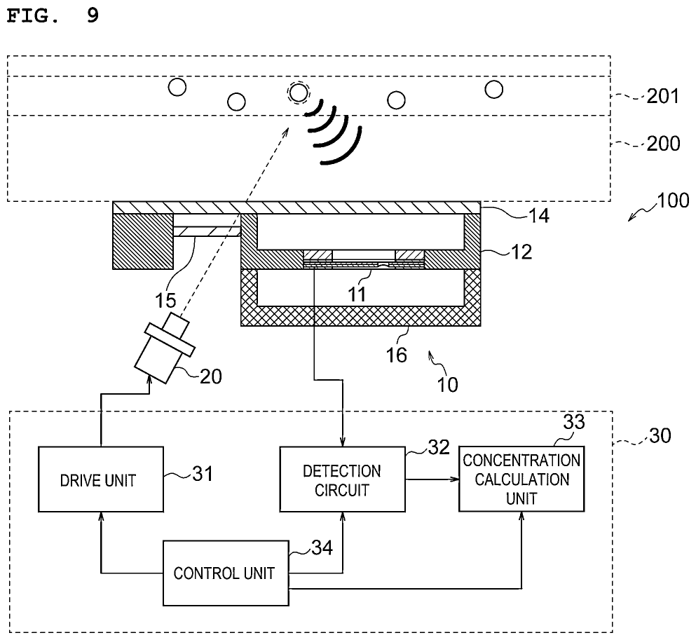

FIG. 8 is a schematic configuration diagram of the measurement device 100 according to the fourth embodiment. As compared with the measurement device 100 according to the first embodiment illustrated in FIG. 1, the seal member 13 is eliminated, and the shape of the housing 12 is changed. An airtight chamber is formed by the housing 12 in a right side of the drawing, and the window portion 15 is fixed in a left side of the drawing. Furthermore, the sensor unit 11 does not include the fixing member 111, and the sensor substrate 112 constitutes a part of a wall surface of the housing 12 without being fitted into the fixing member 111.

Even with such a configuration, a change in air pressure caused by the photoacoustic wave (elastic wave) in the airtight chamber is measured by the sensor unit 11. As a result, the change in the air pressure of the airtight chamber can be easily measured, and therefore, the measurement accuracy of the measurement device 100 can be improved.

### Fifth Embodiment

In the fourth embodiment, in the measurement unit 10, the sensor unit 11 constitutes a part of a wall surface of the housing 12, and the sensor unit 11 separates the airtight chamber from the outside, but the present invention is not limited thereto. In a fifth embodiment, an example in which a closed space is further provided on a side opposite to the airtight chamber of the sensor unit 11 will be described.

FIG. 9 is a schematic configuration diagram of the measurement device 100 according to the fifth embodiment. As compared with the measurement device 100 according to the fourth embodiment illustrated in FIG. 8, a housing 16 is further provided on a side opposite to a measurement target 200 side of the sensor unit 11. Similarly to the case of the housing 12, the interior of the housing 16 is closed to form a closed space.

The sensor unit 11 detects the pressure of the airtight chamber affected by the photoacoustic wave emitted from the measurement target 200, and this is performed by measuring a displacement caused by an air pressure difference in a space facing both directions of the displacement (vertical direction in drawing) by a sensing portion (cantilever). That is, in the first to fourth embodiments, the sensor unit 11 measures the displacement of the sensing portion caused by a pressure difference between the airtight chamber and the outside air. On the other hand, in the fifth embodiment, the magnitude of the photoacoustic wave is measured by measuring the displacement of the sensing portion caused by the pressure difference between the airtight chamber on an upper side in the drawing which is close to the measurement target 200 and is easily affected by the photoacoustic wave and the closed space on a lower side in the drawing which is far from the measurement target 200 and is hardly affected by the photoacoustic wave. The closed space is provided on a side opposite to the airtight chamber of the sensor unit 11, so that the sensing portion is hardly affected by the surroundings than when the sensing portion is in directly contact with the outside air, and as a result, the measurement accuracy of the measurement device 100 can be improved. In the present embodiment, the airtight chamber in the housing 12 and the closed space in the housing 16, which are separated by the sensor unit 11, have substantially the same opening areas and volumes, but the present invention is not limited thereto. Both the airtight chamber and the closed space may have any size.

Various embodiments and modifications may be made without departing from the broad spirit and scope of the present invention. The above-described embodiments are for describing the present invention, and do not limit the scope of the present invention. That is, the scope of the present invention is indicated by the claims rather than the embodiments. Various modifications made within the scope of the claims and within the meaning of the invention equivalent thereto are regarded as within the scope of the present invention.

### REFERENCE SIGN LIST

10 measurement unit
11 sensor unit
12, 16 housing
13 seal member
14 contact portion
15 window portion
20 modulated light source
30 circuit unit
60 thermal light source
100 photoacoustic wave measurement device
200 measurement target
201 blood vessel

1. A photoacoustic wave measurement device comprising:
a modulated light source that irradiates a measurement target with far-infrared light which is subjected to intensity modulation; and
a measurement unit that measures a photoacoustic wave emitted from the measurement target irradiated with the far-infrared light, wherein
the measurement unit includes:
an airtight chamber that is filled with a gas therein and receives the photoacoustic wave emitted from the measurement target; and
a pressure sensor constituting a part of a wall portion that partitions the airtight chamber from the outside.

2. The photoacoustic wave measurement device according to claim 1, wherein
the pressure sensor is constituted by a cantilever having a base end connected to the wall portion and a tip end that is a free end and displaceable around the base end, and including a resistance layer whose resistance value changes according to a displacement of the tip end.

3. The photoacoustic wave measurement device according to claim 1, further comprising:
outside the pressure sensor on a side opposite to the airtight chamber, a closed space that is separated from outside air.

4. The photoacoustic wave measurement device according to claim 1, wherein
the airtight chamber includes a window portion in a portion to be directly irradiated with the far-infrared light from the modulated light source, and
the window portion is made of a material having high transmittance of the far-infrared light.

5. The photoacoustic wave measurement device according to claim 4, wherein
the window portion is made of silicon, germanium, or polyethylene.

6. The photoacoustic wave measurement device according to claim 1, wherein
the airtight chamber is configured to come into contact with the measurement target, and the wall portion that partitions the airtight chamber from the measurement target is made of an elastic substance.

7. The photoacoustic wave measurement device according to claim 6, wherein
the wall portion that partitions the airtight chamber from the measurement target is made of silicon, germanium, or polyethylene.

8. The photoacoustic wave measurement device according to claim 6, wherein
the airtight chamber includes:
a bottomed member with one surface opened;
a contact portion facing an end portion on a side surface of the bottomed member and to be brought into contact with the measurement target; and
a sealing member provided between the contact portion and the end portion on the side surface of the bottomed member.

9. The photoacoustic wave measurement device according to claim 1, wherein
the airtight chamber includes a bottomed member having an opening to be closed by the measurement target.

10. The photoacoustic wave measurement device according to claim 1, further comprising:
a resonator that amplifies a pressure change in the airtight chamber.

11. The photoacoustic wave measurement device according to claim 1, wherein
a plurality of pressure sensors including the pressure sensor are provided, the plurality of pressure sensors are provided at different positions, and frequency bands of pressure changes respectively measurable by the plurality of pressure sensors overlap one another.

12. The photoacoustic wave measurement device according to claim 1, further comprising:
a plurality of pressure sensors including the pressure sensor are provided, and frequency bands of pressure changes respectively measurable by the plurality of pressure sensors are different.

13. The photoacoustic wave measurement device according to claim 1, wherein
the modulated light source includes
a laser light source and a chopper part that performs the intensity modulation, or
a thermal light source including an electrical resistor.

14. A photoacoustic wave measurement system comprising:
a modulated light source that irradiates a measurement target with far-infrared light which is subjected to intensity modulation;
a measurement unit that measures a photoacoustic wave emitted from the measurement target irradiated with the far-infrared light; and
a controller that controls the modulated light source and the measurement unit, wherein
the measurement unit includes:
an airtight chamber that is filled with a gas therein and receives the photoacoustic wave emitted from the measurement target; and
a pressure sensor constituting a part of a wall portion that partitions the airtight chamber from the outside, and
the controller
irradiates the modulated light source with the far-infrared light which is subjected to the intensity modulation,
acquires a pressure change in the airtight chamber by the pressure sensor, and
calculates a concentration of a predetermined component in the measurement target according to the pressure change acquired by the pressure sensor.

15. The photoacoustic wave measurement system according to claim 14, wherein
the controller uses a modulation pattern of the modulated light source to remove noise from the pressure change acquired by the pressure sensor and calculate the concentration of the predetermined component.

16. The photoacoustic wave measurement system according to claim 15, wherein
a method of removing the noise is synchronous detection.
